(19) United States
(12) Patent Application Publication
(10) Pub. No.: US 2017/0326302 A1
(43) Pub. Date: Nov. 16, 2017

(54) Drug Injection Device with Visual End-Of-Contents Indicator System

(71) Applicant: Novo Nordisk A/S, Bagsvaerd (DK)

(72) Inventors: Morten Soerensen, Ballerup (DK); Philip Wulff Eckert, Virum (DK); Christian Peter Enggaard, Vejby (DK); Brian Jensen, Broenshoej (DK)

(73) Assignee: Novo Nordisk A/S, Bagsvaerd (DK)

(21) Appl. No.: 15/534,514

(22) PCT Filed: Jan. 8, 2016
(86) PCT No.: PCT/EP2016/050262

§ 371 Date: Jun. 9, 2017

(30) Foreign Application Priority Data
Jan. 8, 2015 (EP) 15150485.9

(51) Int. Cl. A61M 5/315; A61M 5/24; A61M 5/31

(52) U.S. Cl. CPC A61M 5/31541; A61M 5/31593; A61M 5/31553; A61M 5/31568; A61M 5/24; A61M 5/3157; A61M 2005/3126; A61M 2205/582; A61M 2205/583

(57) ABSTRACT

Medical device (1) for injecting a set dose of an injectable product into a patient, the device having a cartridge containing the injectable product, a movable dosing driver (11) and a movable dose setting driver (15), and an end-of-contents indicator system (13) having counting means (17) with a counting part (19), and indication means (21) with a signal part (23), wherein the counting part (19) and the signal part (23) are formed with cooperating end-of-contents contact faces (25′, 25″), wherein the signal part (23) is movable between a first indication position and a second indication position, wherein the signal part (23) comprises a biasing means (33) for biasing the signal part (23) towards the first indication position, wherein the counting part (19) comprises a coupling (26), said coupling (26) is configured for operably connecting the counting part (19) to the dose setting driver (15), such that moving of the dose setting driver(15) towards increasing the set dose moves the contact face (25′) of the counting part (19) in relation to the contact face (25″) of the signal part (23) towards engagement of the contact surfaces (25′, 25″), and such that moving of the dose setting driver (15) towards decreasing the set dose moves the contact face (25′) of the counting part (19) away from the contact face (25″) of the signal part (23), wherein the counting part (19) is configured for providing engagement of the contact faces (25′, 25″) when the set dose reach the end-of-contents in the cartridge, and wherein the counting part (19) is configured for moving the signal part (23) towards the second indication position when the set dose is exceeding the end-of-contents in the cartridge. This way, if the user tries to dial a dose of drug beyond the amount remaining in the cartridge, an additional indication is given to the user via a distinctive area (41/43) of the signal part (23) becoming visible through a window (7) of the injector.

The current invention relates to an end-of-contents indicator system, for a medical device for injecting a set dose of an injectable product into a patient.

### BACKGROUND OF THE INVENTION

In relation to medical devices, more specifically self-administered drug injection delivery devices, for example devices used to inject insulin from a cartridge in a patient's body, it is crucial that the devices are used in the intended manner in order to avoid any risk of improper treatment and thereby potentially compromise the health of the patient.

Therefore it is of vital importance that the devices are easy and intuitive to use such that they provide the patient with the ability to use the device as intended.

To reduce the risk of improper use the devices can feature fail-safe mechanisms to perform the various functions of the devices.

Devices that can be used for multiple injections of a set or adjustable doze are known as multi-dose devices.

In all multi-dose devices it is relevant to indicate to the user when the cartridge is approaching the end-of contents in order to avoid injecting an incomplete dose as the cartridge become empty.

An object of the present invention is to provide a device that provides clear indication when a dose that is exceeding the remaining dose amount is set on the injection device.

### SUMMARY OF THE INVENTION

According to the present invention, this is achieved by an end-of-contents indicator system for a medical device for injecting a set dose of an injectable product into a patient, the device having a cartridge containing the injectable product, a movable dosing driver and a movable dose setting driver, the system comprising;

a) a counting means with a counting part, and

b) an indication means with a signal part,

wherein the counting part and the signal part are formed with co-operating end-of-contents contact faces, wherein the signal part is movable between a first indication position and a second indication position, wherein the signal part comprises a biasing means for biasing the signal part towards the first indication position, wherein the counting part comprises a coupling, said coupling is configured for operably connecting the counting part to the dose setting driver, such that moving of the dose setting driver towards increasing the set dose moves the contact face of the counting part in relation to the contact face of the signal part towards engagement of the contact surfaces, and such that moving of the dose setting driver towards decreasing the set dose moves the contact face of the counting part away from the contact face of the signal part, wherein the counting part is configured for providing engagement of the contact faces when the set dose reach the end-of-contents in the cartridge, and wherein the counting part is configured for moving the signal part towards the second indication position when the set dose is exceeding the end-of-contents in the cartridge.

It is herewith achieved that an injection device is provided with clear indication when a user sets a dose that is exceeding the remaining contents in the cartridge.

The end-of-contents indicator system is suitable for a medical device for injecting a set dose of an injectable product into a patient. The device is of a type comprising a cartridge, a dosing driver and a dose setting driver.

A full cartridge is inserted into the injection device. Preferably the cartridge has a capacity such that a number of doses can be administered before the cartridge reaches the end-of-contents, where it is empty.

The dosing driver is movable and used to move a piston in the cartridge to expel the injectable product through a syringe connected to the cartridge during injection of the injectable product.

The dose setting driver is movable and operably connected to a dose setting mechanism. The dose setting driver can be moved towards increasing or decreasing the set dose.

Such devices include self-administered drug injection delivery devices, for example devices used to inject insulin. With such devices the user is capable of completing an injection without the assistance of a medical professional.

The end-of-contents indicator system comprises a counting means with a counting part and an indication means with a signal part.

The counting means tracks the remaining contents in the cartridge. The counting means comprises a counting part. The counting part is coupled to the dose setting driver through a coupling. The counting part moves in r

The indication means provides indication when a set dose exceeds the remaining contents in the cartridge. The signal part is movable between a first indication position and a second indication position.

The first indication position is the default position as the signal part comprises biasing means that bias the signal part towards the first position. The first position indicates that the set dose does not exceed the remaining contents in the cartridge.

The second position indicates that the set dose exceeds the remaining contents in the cartridge, i.e. that the end-of-contents is exceeded.

The end-of-contents contact faces engage when the set dose reaches the end-of-contents. Upon further movement of the dose setting driver the counting part moves further and thereby moves the signal part to its second indication position.

If the dose setting driver is moved to decrease the set dose firstly to the end-of-contents the counting part returns to a point where the end-of-contents contact faces are just engaged. The biasing means returns the signal part to its first indication position to indicate the set dose is equal to or below the end-of-contents. If the dose setting driver is moved further to decrease the set dose the counting part and signal part becomes disengaged.

This invention makes it possible to indicate to the user if there is not enough injectable product left in the cartridge to deliver the set dose.

Moreover, the mechanism makes it possible for the user to dial down to the remaining dose, after getting the last full dose, in order to use the total content of the cartridge.

The visual indicator makes it easy for the user to identify if the cartridge contains enough injectable product to complete the set dose.

The invention can be combined with a mechanism that automatically resets the set dose when the dose delivery process is finalised, e.g. while mounting the device cap.

The invention may further be combined with a mechanism that disables activation of the dose activation mechanism, e.g. by blocking a dose activation button from being pressed. By adding this mechanism an additional tactile indicator can be introduced, i.e. the user can sense that the button can not be pressed and it is ensured that the device can not deliver any dose if there is not enough injectable product in the cartridge to deliver the set dose.

Hereby ensuring fail-safe operation should the user ignore a visual indication that the set dose exceeds the end-of-contents.

In another aspect of the invention the object is achieved by a drug delivery device comprising: a housing, a variable volume reservoir adapted to hold a drug and having a predetermined pre-use capacity, a dose setting mechanism operable to set a dose to be expelled from the variable volume reservoir, a drug expelling mechanism operable to expel the dose, a counting element for logging an accumulated dose expelled from the variable volume reservoir, the counting element being operatively coupled with the dose setting mechanism and configured to move in a first direction relative to the housing in response to a dose increasing dose setting operation and to move in a second direction relative to the housing in response to a dose decreasing dose setting operation, a signal element configured to interact with the counting element and move relative to the housing from a first indicating position to a second indicating position in response to the counting element surpassing an end-of-content position, the end-of-content position being reached by an accumulated movement of the counting element in the first direction which corresponds to the accumulated dose expelled from the variable volume reservoir equalling the predetermined pre-use capacity, and a display arrangement for visual inspection of the signal element in at least one of the first indicating position and the second indicating position.

The signal element may further be configured to move from the second indicating position to the first indicating position in response to the counting element moving back to the end-of-content position to thereby indicate when during a dose decreasing dose setting operation the maximum injectable dose is reached.

The drug delivery device may further comprising dose blocking means configured to disable the drug expelling mechanism in response to the counting element surpassing the end-of-content position (during movement in the first direction) to prevent an expelling of drug when the set dose exceeds the dose remaining in the variable volume reservoir.

It should be emphasized that the term “comprises/comprising/comprised of” when used in this specification is taken to specify the presence of stated features, integers, steps or components but does not preclude the presence or addition of one or more other features, integers, steps, components or groups thereof.

### BRIEF DESCRIPTION OF THE DRAWINGS

The invention will be explained in more detail below with reference to the accompanying drawing, where:

FIG. 1 shows a perspective view of a first embodiment of a medical device for injecting a set dose of an injectable product into a patient, wherein the set dose is 0 units,

FIG. 2 shows a perspective view of the medical device in FIG. 1, wherein the set dose is 40 units,

FIG. 3 shows a perspective view of a first embodiment of an end-of-contents indicator system,

FIG. 4 shows a section view through section A-A of FIG. 3,

FIG. 5 FIG. 5 shows a perspective view of the end-of-contents indicator system of FIG. 3,

FIG. 6 shows a perspective view of the counting part of the end-of-contents indicator system of FIG. 3,

FIG. 7 shows a perspective view of the signal part of the end-of-contents indicator system of FIG. 3,

FIG. 8 shows a perspective view of a second embodiment of a medical device for injecting a set dose of an injectable product into a patient,

FIG. 9 shows a perspective view of a second embodiment of an end-of-contents indicator system,

FIG. 10 shows a section view of the end-of-contents indicator system through section A-A of FIG. 9,

FIG. 11 shows a section view of the end-of-contents indicator system through section B-B of FIG. 10,

FIG. 12 shows a first perspective view of the dose setting driver of the end-of-contents indicator system of FIG. 9,

FIG. 13 shows a perspective view of the counting part of the end-of-contents indicator system of FIG. 9,

FIG. 14 shows a first perspective view of the signal part of the end-of-contents indicator system of FIG. 9,

FIG. 15 shows a second perspective view of the signal part of the end-of-contents indicator system of FIG. 9,

FIG. 16 shows a perspective view of a combination of an end-of-contents indicator system with an injection device, where the set dose is less than the end-of-contents, and

FIG. 17 shows a perspective view of the combination of FIG. 16, where the set dose exceeds the end-of-contents.

In the following, the invention will be described in greater detail with reference to embodiments shown by the enclosed figures. It should be emphasized that the embodiments shown are used for example purposes only and should not be used to limit the scope of the invention.

### DETAILED DESCRIPTION OF THE EMBODIMENTS

In the explanation of the figures, identical or corresponding elements will be provided with the same designations in different figures. Therefore, no explanation of all details will be given in connection with each single figure/embodiment.

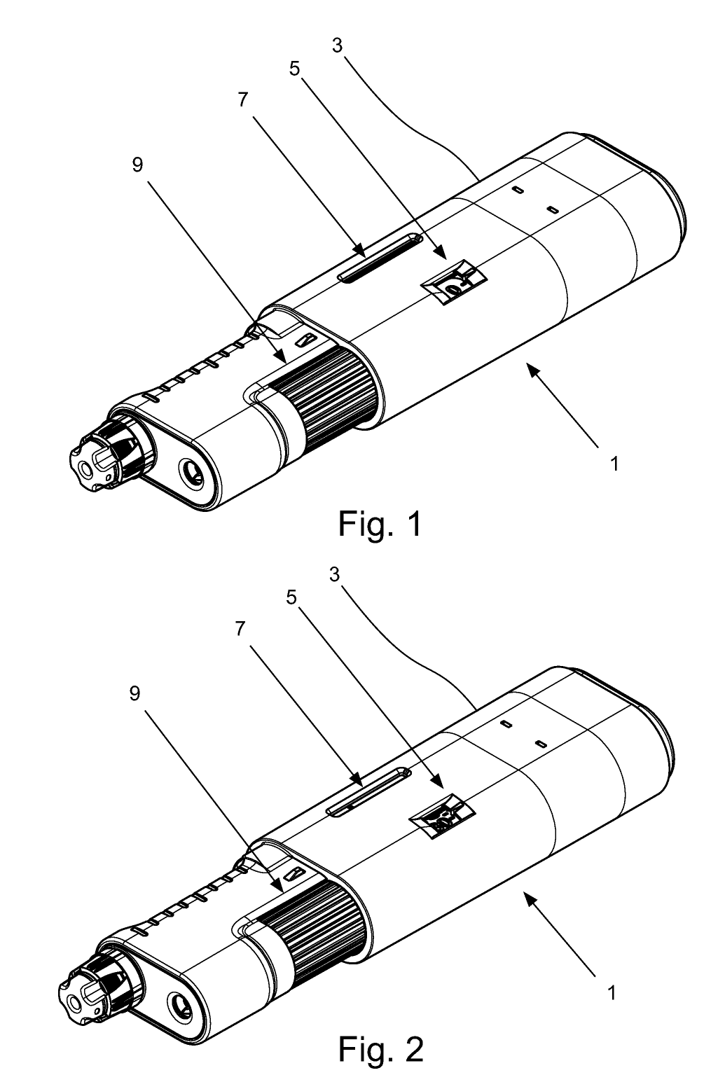

FIGS. 1 and 2 each shows a perspective view of a first embodiment of a medical device 1 for injecting a set dose of an injectable product into a patient. In FIG. 1 the dose is 0 units and in FIG. 2 the set dose is 80 units.

The medical device 1 comprises a housing 3 holding a cartridge (not shown) with an injectable product. The housing 3 has a dose window 5, where the set dose is displayed, and an end-of-contents indicator window 7, where a signal is displayed that indicates whether or not the set dose exceeds the contents in the cartridge, i.e. exceeds the end-of-contents.

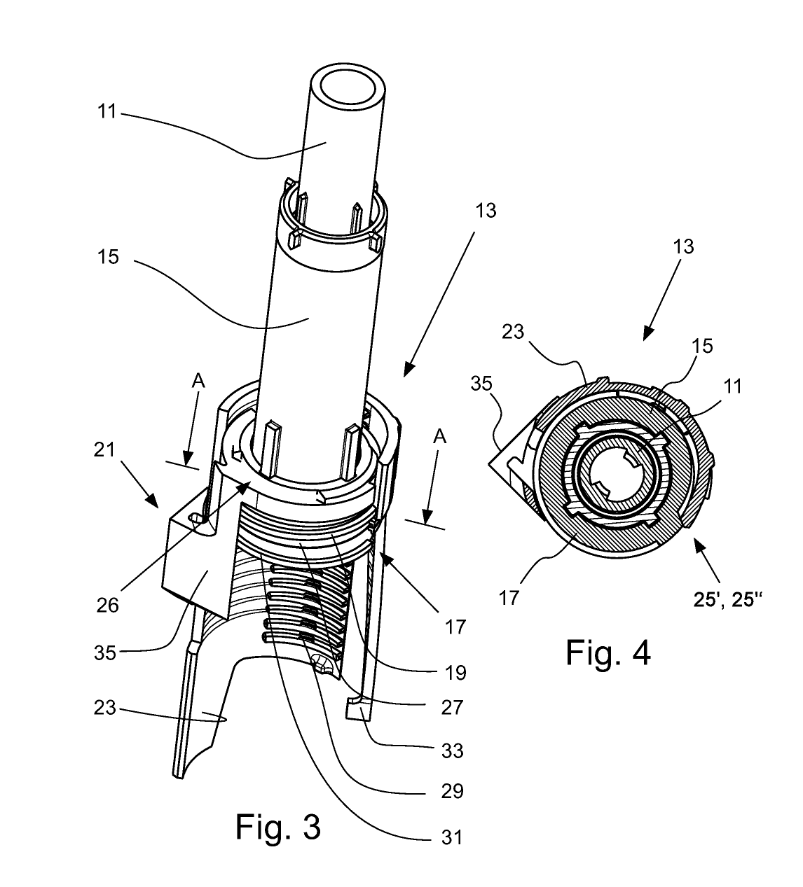

The set dose can be adjusted by operating a dosing dial 9. The dosing dial 9 is operably connected to the dosing driver 11, see FIG. 3.

In FIG. 1 the set dose is 0 units. Therefore the set dose does not exceed the end-of-contents. This is visually indicated in the end-of-contents indicator window 7. In FIG. 1 the indication is provided with a solid black colour. In other embodiments other colours may be applied.

In FIG. 2 the set dose is 80. In this example the set dose is exceeding the end-of-contents and the visual indication in the end-of-contents indicator window 7 has changed from the indication in FIG. 1. The indication is provided with a white colour. In other embodiments other colours may be applied.

FIGS. 3 to 7 shows a first embodiment of an end-of-contents indicator system 13.

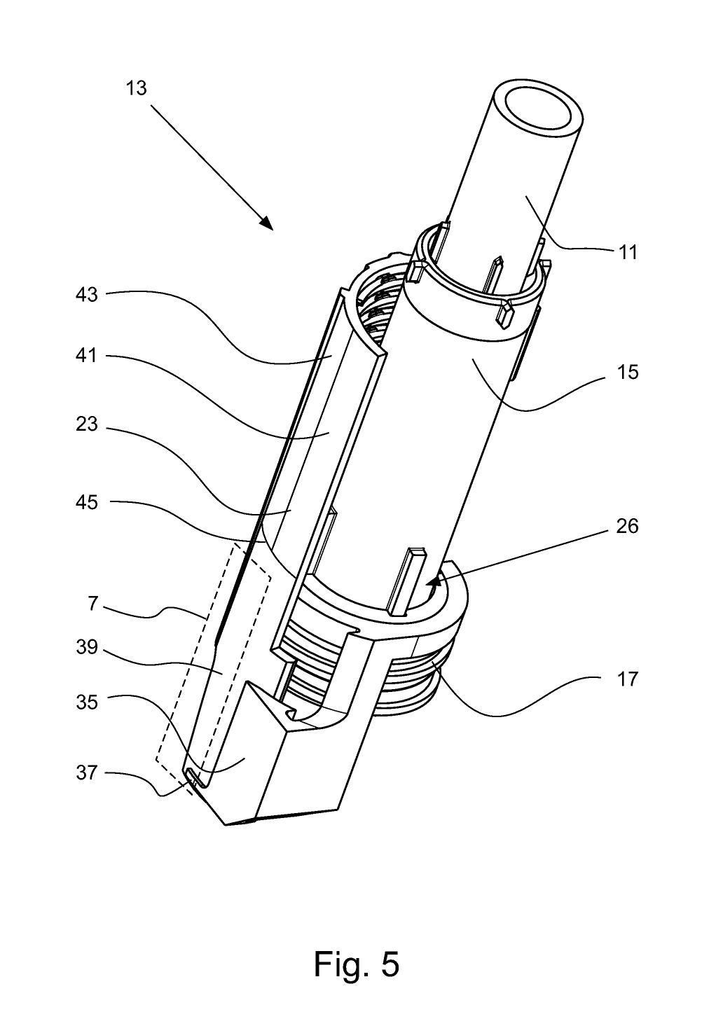

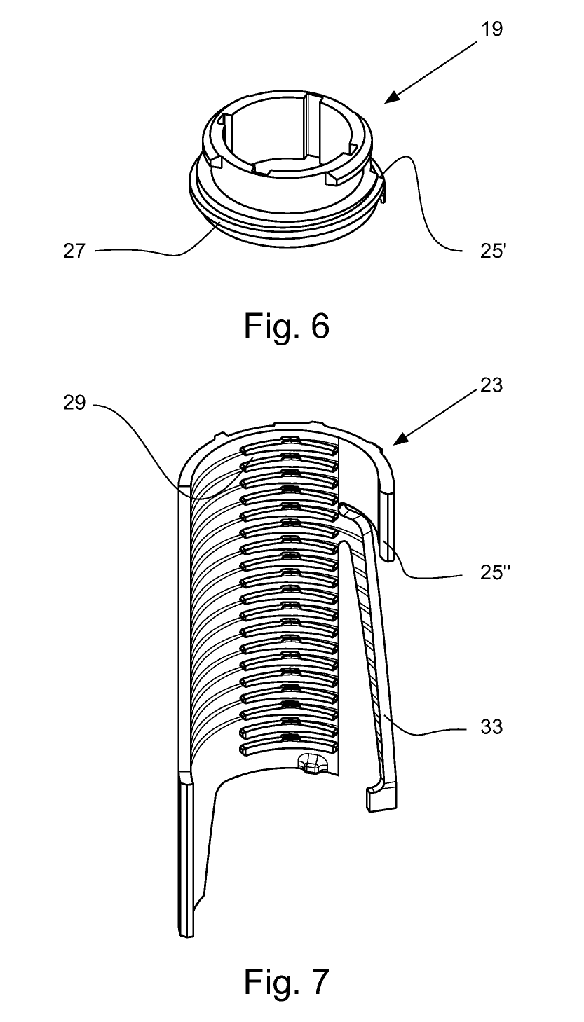

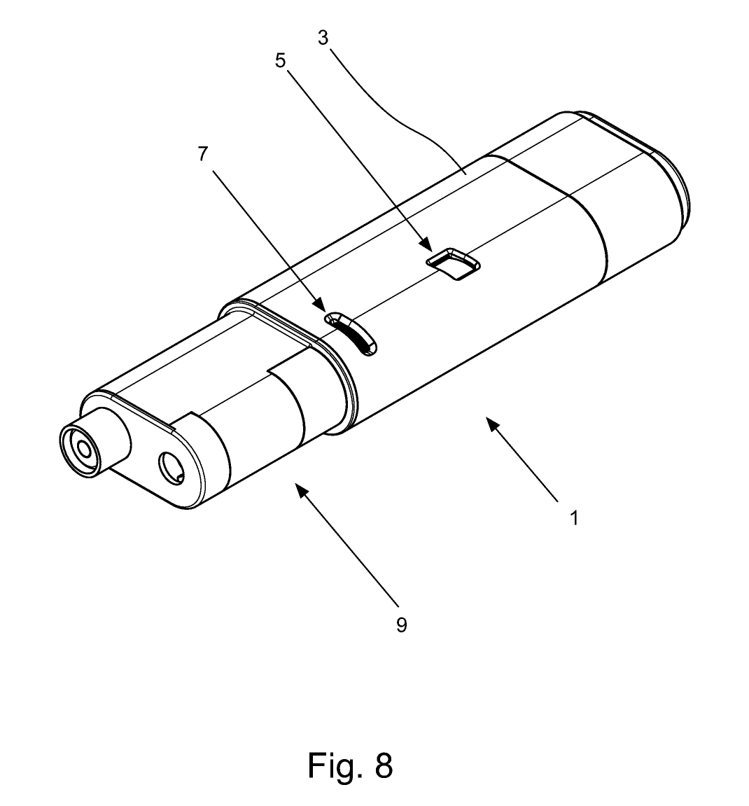

FIG. 3 shows a perspective view of the end-of-contents indicator system 13 in a position where the set dose is at the end-of-contents of the cartridge (not shown) and FIG. 4 shows a section view through section A-A of FIG. 3. FIG. 5 shows a perspective view of the end-of-contents indicator system 13 in a position where dosing, i.e. injection of the injectable product has been completed. FIGS. 6 and 7 shows a perspective view of the counting part 19 and the signal part 23, respectively.

Supports and bearings for the various parts are not shown for clarity. In use the end-of-contents indicator system 13 is installed in the medical device 1 that includes the required bearings and supports to secure the end-of-contents indicator system 13 within the medical device 1.

The end-of-contents indicator system 13 is in communication with a movable dosing driver 11 and a movable dose setting driver 15.

In the first embodiment the dosing driver 11 is placed inside the dose setting driver 15 in a coaxial configuration. The dosing driver 11 and the dose setting driver 15 can rotate independently of each other.

The end-of-contents indicator system 13 comprises a counting means 17 with a counting part 19 and an indication means 21 with a signal part 23.

The counting part 19 and the signal part 23 are formed with co-operating end-of-contents contact faces 25′, 25″.

The counting part 19 and the dose setting driver 15 is operably connected through a coupling 26 in the form of a spline joint, where grooves are formed in the counting part 19 and teeth are formed on the dose setting driver 15.

The counting part 19 is formed with an external thread 27 and the signal part 23 is formed with a left and right wound overlapping internal thread segment 29. The external thread 27 of the counting part 19 communicates with the right wound internal thread segment 29 of the signal part 23. Moreover the dosing driver 11 is formed with and external thread 31 that is in communication with the left wound external thread segment 29 of the signal part 23.

In the embodiment shown the counting part 19 is a circular ring.

The signal part 23 comprises a biasing means 33 in the form of a spring arm, that is in contact with an abutment surface (not shown) on the housing, such that the signal part 23 is rotationally locked in relation to the housing until the set dose exceed the end-of-contents.

The indication means 21 comprises a dose size indicator 35. The dose size indicator 35 is axially locked to the counting part 19 and rotationally locked in relation to the signal part 23.

The dose size indicator 35 has a pointer 37 for indicating the set dose size in relation to a scale on the signal part 23.

In FIG. 5 the scale is shown. The location of the end-of-contents indicator window 7 in the housing 3, see FIGS. 1 and 2, is shown with a dashed line.

The scale has three indication zones. The first indication zone 39 and the second and third indications zones 41, 43 respectively, are divided by an.

The first indication zone 39 indicates that the set dose does not exceed the remaining contents in the cartridge. The end-of-contents indication line 45 indicates the point where the cartridge is empty. The second indication zone 41 indicates that there is enough injectable product left in the cartridge to complete the set dose. The third indication zone 43 indicates that the end-of-contents will be exceeded with the set dose. The functioning of the scale will be explained further below.

During dose setting the dose setting driver 15 is rotated to set the desired dose size. The dosing driver 11 is stationary during dose setting.

The signal part 23 is fixed rotationally and axially due to the biasing means 33 and the threaded connection to the stationary dosing driver 11.

As the counting part 19 is rotationally locked to the dose setting driver 15, the counting part 19 is rotating with the dose setting driver 15. The counting part 19 travels in the axial direction due to its threaded interface 27, 29 with the stationary signal part 23.

When the dose setting driver 15 is rotated counter clockwise the counting part 19 travels towards engagement between the co-operating end-of-contents contact faces 25′, 25″.

When the dose setting driver 15 is rotated clockwise the counting part 19 travels away from engagement between the co-operating end-of-contents contact faces 25′, 25″.

The dose size indicator 35 travels with the counting part 19 and indicate with its pointer on the scale, the set dose size.

During dosing the dosing driver 11 will rotate and the dose setting driver 15 remains stationary.

The signal part 23 translates in the axial direction due to the threaded interface with the dosing driver 11. This will also drive the counting part 19 in the same direction as the signal part 23.

The pitch of the left and right wound threads 29 is identical. Therefore the counting part 19 will return to its original axial position in relation to the housing/frame whenever the dosing of the set dose has been completed. However, the signal part 23 will have travelled relative to the counting part 19 and thereby the housing/frame upon completion of the dosing. The travelled distance corresponds to the dose amount that has just been outdosed.

The end-of-contents indication line 45 becomes visible in the end-of-contents indicator window 7. As more doses have been administered the end-of-contents indication line 45 will appear to travel across the end-of-contents indicator window 7 and the second indication zone 41 of the scale becomes visible.

This mechanism enables a visual indication of the residual amount of injectable product left in the cartridge, the amount of injectable product used and the relation to the set dose.

From this mechanism it can be seen that there is not enough drug left when the end-of-contents indication line 45 is below the dose size indicator.

As the signal part 23 travels in relation to the counting part 19 it reaches a point where the end-of-contents contact faces 25′, 25″ meet. The signal part 23 will rotate to its second indication position and the third indication zone 43 becomes visible in the end-of-contents window 7 to indicate that the set dose exceeds the end-of-contents. At this point the signal part 23 make contact with a stop (not shown) on the housing that prevents further rotation of the signal part 23.

The contact faces 25′, 25″ are angled. Therefore upon further turning of the dose setting driver 15 beyond the end-of-contents point, the angled contact face 25′ of the counting part 19 forces the contact face 25″ of the signal part 23 outwards. The signal part 23 deforms and allows further turning of the counting part 19, while maintaining the third indication zone 43 visible.

When the dose setting driver 15 is returned to a point where the set dose is below the end-of-contents the signal part 23 will return to its first indication position assisted by the biasing means 33.

FIG. 8 shows a perspective view of a second embodiment of a medical device 1 for injecting a set dose of an injectable product into a patient.

The medical device 1 comprises a housing 3 holding a cartridge (not shown) with an injectable product. The housing 3 has a dose window 5, where the set dose is displayed, and an end-of-contents indicator window 7, where a signal is displayed that indicates whether or not the set dose exceeds the contents in the cartridge, i.e. exceeds the end-of-contents.

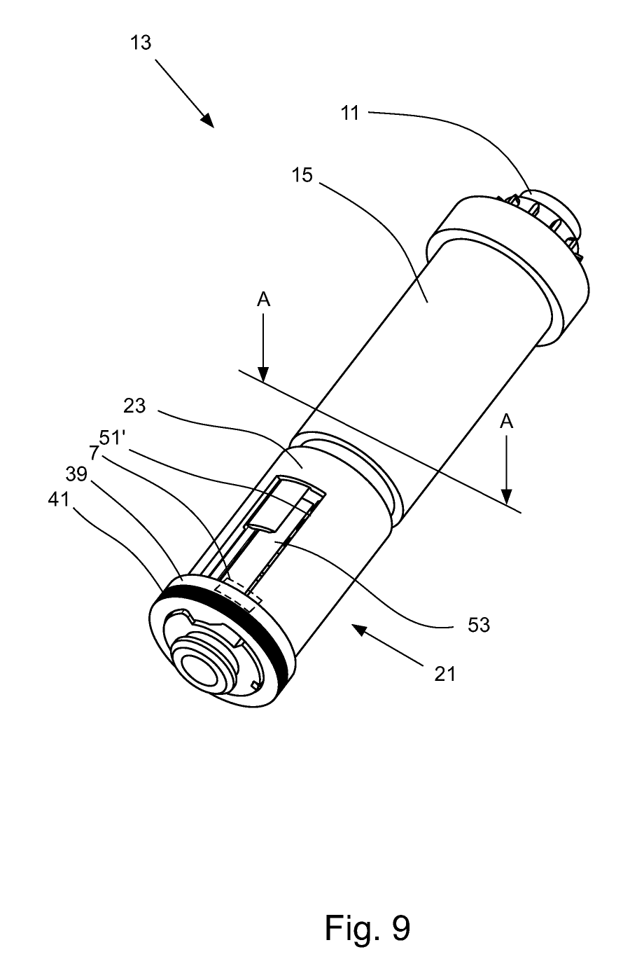

The set dose can be adjusted by operating a dosing dial 9. The dosing dial 9 is operably connected to the dosing driver 11, see FIG. 9.

FIGS. 9 to 15 shows a second embodiment of an end-of-contents indicator system 13.

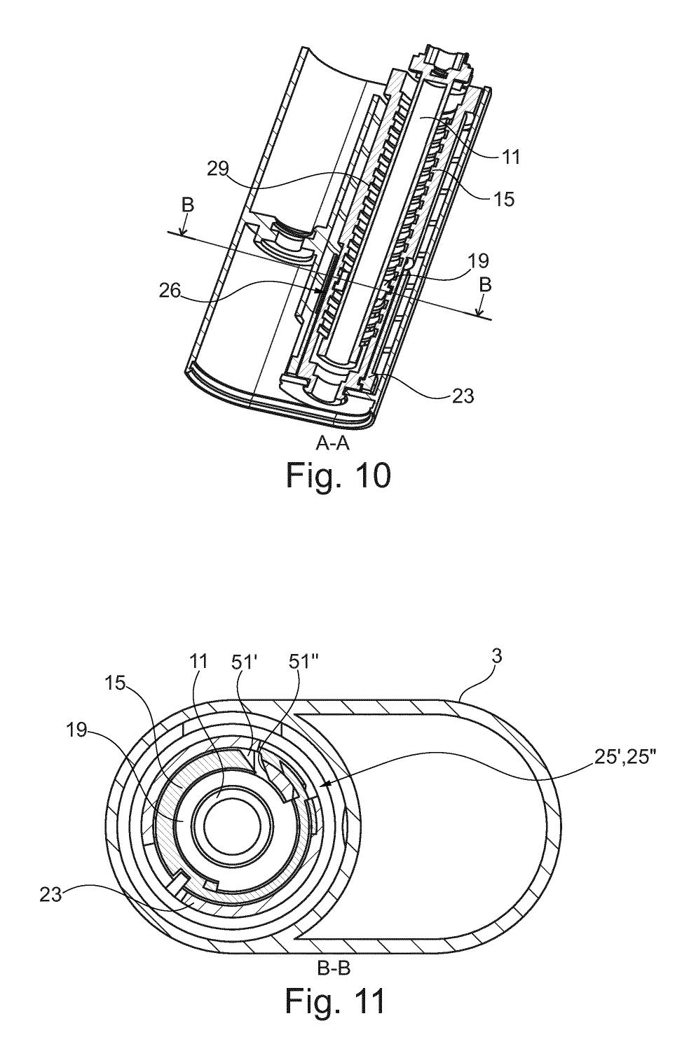

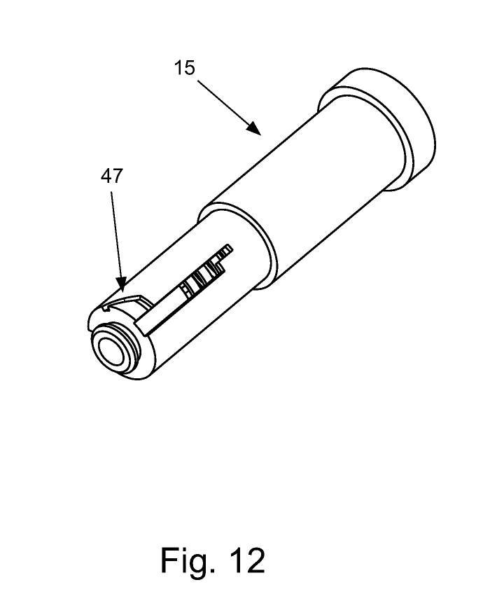

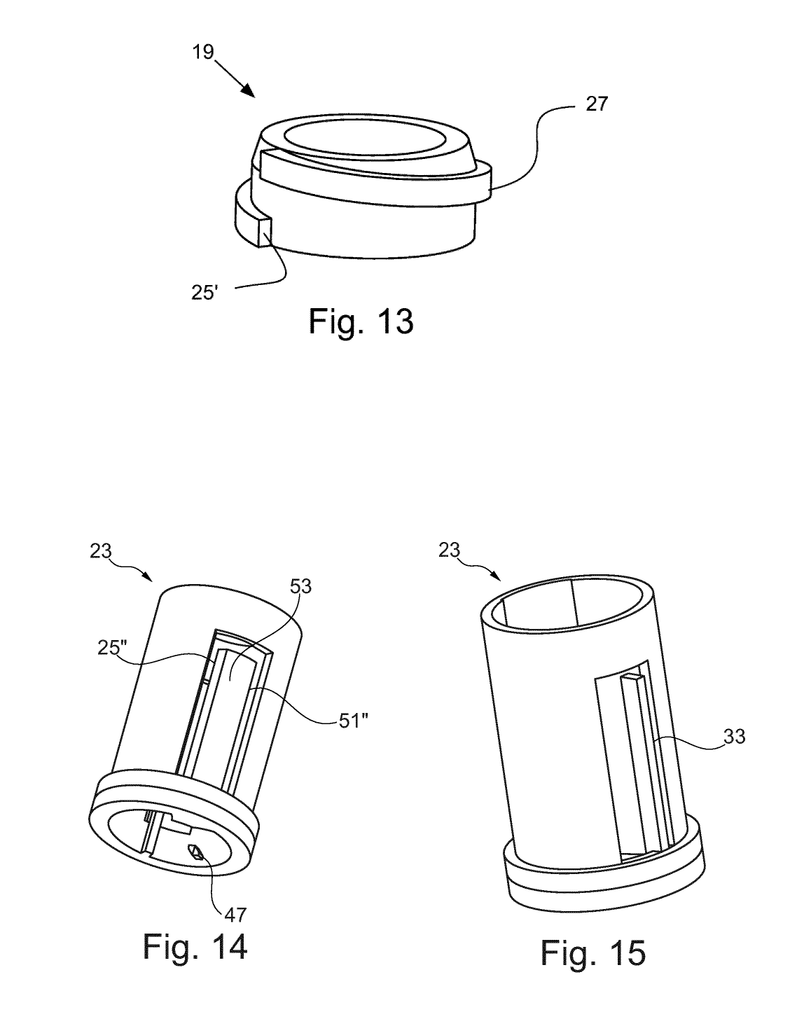

FIG. 9 shows a perspective view of a second embodiment of an end-of-contents indicator system 13. FIG. 10 shows a section view of the end-of-contents indicator system 13 through section A-A of FIG. 9. FIG. 11 shows a section view of the end-of-contents indicator system 13 through section B-B of FIG. 10. FIG. 12 shows a perspective view of the dose setting driver 15. FIG. 13 shows a perspective view of the counting part 19. FIGS. 14 and 15 shows two perspective views of the signal part 23.

Supports and bearings for the various parts are not shown for clarity. In use the end-of-contents indicator system 13 is installed in the medical device 1 that includes the required bearings and supports to secure the end-of-contents indicator system 13 within the medical device 1.

The end-of-contents indicator system 13 shown in FIG. 9 is in communication with a movable dosing driver 11 and a movable dose setting driver 15.

In the second embodiment the dosing driver 11 is placed inside the dose setting driver 15 in a coaxial configuration. The dosing driver 11 and the dose setting driver 15 are rotatable.

The end-of-contents indicator system 13 comprises a counting means 17, see FIG. 10 with a counting part 19 and an indication means 21 with a signal part 23.

The counting part 19 and the signal part 23 are formed with co-operating end-of-contents contact faces 25′, 25″.

The counting part 19 and the dose setting driver 15 are operably connected through a coupling 26 in the form of a threaded coupling. The counting part 19 has an external thread 27 and the dose setting driver has an internal thread 29.

The dosing driver 11 and the counting part 19 are formed with a co-operating spline coupling, such that the dosing driver 11 is rotationally locked to the counting part 19.

In the embodiment shown the counting part 19 is a circular ring.

The signal part 23 comprises a biasing means 33 in the form of a spring arm, that is in contact with the dose setting driver 15 such that the signal part 23 is rotationally locked in relation to the dose setting driver 15 until the end-of-contents is reached. Hereafter the dose setting driver rotates 15 in relation to the signal part 23 and the biasing means 33 is tensioned.

During dose setting the counting part 19 is not rotating because it is rotationally locked to the stationary dosing driver 11. The counting part 19 is translating axially due to the threaded coupling with the dose setting driver 15.

The signal part 23 provides a binary indication in relation to the end-of-contents. The end-of-contents indicator window 7 is indicated in FIG. 9 with a dashed line.

The signal part 23 has a cylindrical first indication zone 39 and a cylindrical second indication zone 41. The signal part 23 changes indication by translating in the axial direction between the first and second indication position.

The axial position of the signal part 23 is defined by a pin 47 inside the signal part 23 that is engaged in an external guideway 49 in the dose setting driver 15.

Hereby relative rotation between the signal part 23 and the dose setting driver 15 will move the signal part 23 in the axial direction to the second indication position that indicates that the set dose exceeds the remaining contents in the cartridge.

At a defined amount of accumulated doses corresponding to the total quantity of injectable product the counting part 19 will have translated to an axial position where the end-of-contents contact faces 25′, 25″ engage.

The counting part 19 is rotationally locked during dose setting. The engagement between the contact faces 25′, 25″ stops the rotation of the signal part 23 in relation to the dose setting driver 15. If the dose setting is further increased the dose setting driver 15 rotates further, resulting in a relative rotation between the signal part 23 and the dose setting driver 15 and axial movement of the signal part 23 due to the co-operation between the pin 47 inside the signal part 23 and external guideway 49 in the dose setting driver 15. This results in a change of the end-of-contents indication.

The signal part 23 and the dose setting driver 15 have angled contact faces 51′, 51″. The contact face 51″ on the signal part 23 is located in a spring arm 53. The contact faces 51′, 51″ engages when the dose setting driver 15 is rotated further.

Even further rotation of the dose setting driver 15 will cause the contact face 51′ on the dose setting driver 15 to bend the spring arm 53 radially outwards. When the spring arm 53 is bent out the relative rotation between the signal part 23 and the dose setting driver 15 will stop. The signal part 23 will rotate with the dose setting driver 15 hereafter if the dose setting is further increased.

If the dose setting is reduced to less than the end-of-contents the spring arm 53 arm returns to its original position. The biasing means 33 returns the signal part 23 to its first indication position.

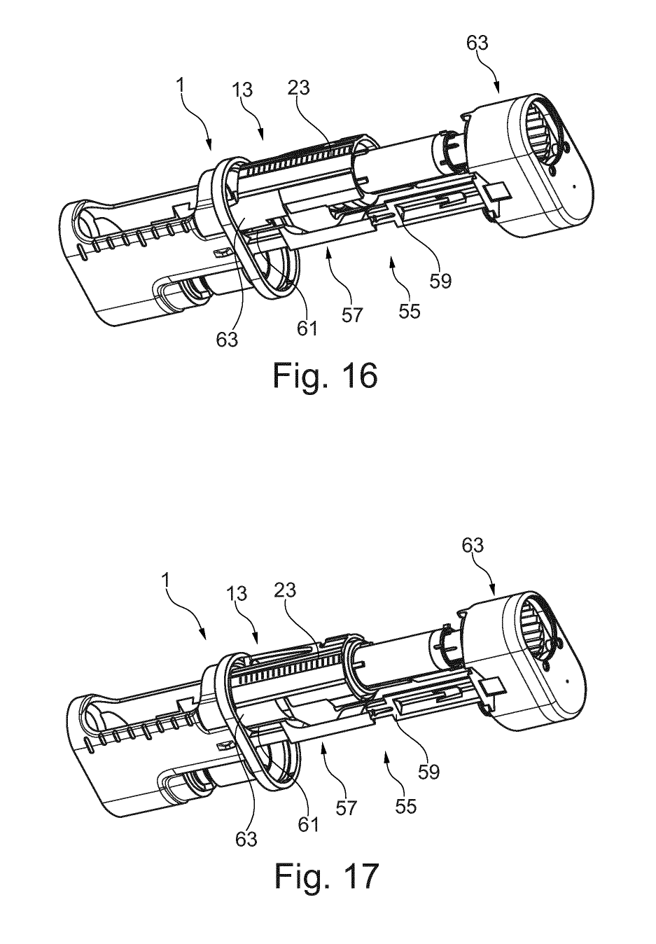

FIGS. 16 and 17 shows a perspective view of a combination 55 of an end-of-contents indicator system 13 with an injection device 1

In FIG. 16 the set dose is less than the end-of-contents. Consequently the signal part 23 is in its first indication position. In FIG. 17 the set dose exceeds the end-of-contents. Consequently the signal part 23 is in its second indication position.

The combination 55 comprises a blocking means 57, for preventing activation of the activation mechanism 63. The blocking means 57 is in communication with the end-of-contents indicator system 13. The blocking means 57 is configured for preventing activation when the signal part 23 is in its second indication position.

The blocking means 57 comprises a link 59 with a resilient blocking arm 61. The end of the blocking arm 61 is axially locked. When the activation mechanism 63 is activated the link 59 moves axially. The blocking arm 61 cannot move axially with the link 59 and must therefore flex inwardly. The link 59 intersects the rotational path of the signal part 23.

The signal part 23 comprises a blocking element 65. The blocking element 65 is away from the blocking arm 61 when the signal part is in its first indication position. In this position the blocking arm 61 is free to flex inside the rotational path of the signal part 23.

When the set dose exceeds the end-of-contents the signal part 23 rotates to its second indication position. The blocking element 65 prevents the blocking arm 61 from flexing inwardly. The link 59 cannot move then and therefore the activation mechanism 63 cannot be activated.

It is to be noted that the figures and the above description have shown the example embodiments in a simple and schematic manner. The internal electronic and mechanical details have not been shown since the person skilled in the art should be familiar with these details and they would just unnecessarily complicate this description.

1. End-of-contents indicator system for a medical device for injecting a set dose of an injectable product into a patient, the device having a cartridge containing the injectable product, a movable dosing driver and a movable dose setting driver, the system comprising:
a) a counting structure with a counting part, and
b) an indication structure with a signal part,
wherein the counting part and the signal part are formed with co-operating end-of-contents contact faces,
wherein the signal part is movable between a first indication position and a second indication position,
wherein the signal part comprises a biasing structure for biasing the signal part towards the first indication position,
wherein the counting part comprises a coupling, said coupling is configured for operably connecting the counting part to the dose setting driver, such that moving of the dose setting driver towards increasing the set dose moves the contact face of the counting part in relation to the contact face of the signal part towards engagement of the contact surfaces, and such that moving of the dose setting driver towards decreasing the set dose moves the contact face of the counting part away from the contact face of the signal part,
wherein the counting part is configured for providing engagement of the contact faces when the set dose reach the end-of-contents in the cartridge, and
wherein the counting part is configured for moving the signal part towards the second indication position when the set dose is exceeding the end-of-contents in the cartridge.

2. End-of-contents indicator system according to claim 1, wherein the counting part is a circular ring.

3. End-of-contents indicator system according to claim 1, wherein the coupling is selected among spline couplings or threaded couplings.

4. End-of-contents indicator system according to claim 2, wherein the ring has an external thread and an internal spline interface.

5. End-of-contents indicator system according to claim 1, wherein the indication structure comprises a dose size indicator, wherein the dose size indicator is coupled to the counting part, for displaying the dose size.

6. End-of-contents indicator system according to claim 1, wherein the signal part is formed with a left wound and a right wound external thread segment.

7. End-of-contents indicator system according to claims 1, wherein the signal part and the dose setting driver is formed with a co-operating pin and guideway.

8. A combination of an end-of-contents indicator system according to claim 1 with a medical device for injecting a set dose of an injectable product into a patient, the device having a cartridge containing the injectable product, a movable dosing driver and a movable dose setting driver and an activation mechanism, for initiating the injection.

9. The combination according to claim 8, wherein the combination comprises a blocking structure, for preventing activation of the activation mechanism, wherein the blocking structure is in communication with the end-of-contents indicator system, wherein the blocking structure is configured for preventing activation when the signal part is in its second indication position.

10. The combination according to claim 9, wherein the blocking structure comprises an axially movable link that is in communication with the activation mechanism, wherein the link comprises a resilient blocking arm with a fixed free end, wherein the signal part comprises a blocking element that is adjacent the blocking arm when the signal part is in its second indication position.
